(19) United States
(12) Patent Application Publication
(10) Pub. No.: US 2025/0134413 A1
(43) Pub. Date: May 1, 2025

(54) MEDICAL SYSTEM AND METHOD

(71) Applicant: Aesculap AG, Tuttlingen (DE)

(72) Inventors: Michael Utz, Tuttlingen (DE); Allan Maas, Konstanz (DE)

(21) Appl. No.: 18/694,159

(22) PCT Filed: Sep. 23, 2022
(86) PCT No.: PCT/EP2022/076584

§ 371 Date: Mar. 21, 2024

(30) Foreign Application Priority Data
Sep. 27, 2021 (DE) 10 2021 124 873.6

(51) Int. Cl. A61B 5/11; A61B 5/00; G06T 7/00; G06T 7/20

(52) U.S. Cl. CPC A61B 5/1121; A61B 5/1126; A61B 5/6801; A61B 5/7264; A61B 5/7435; G06T 7/0012; G06T 7/20; A61B 2562/0219; G06T 2207/10116; G06T 2207/30008

(57) ABSTRACT

A medical system and method are used for determining kinematics of bones. The system includes a capturing unit for creating an image of bones in a defined orientation; a data-processing unit that provides an output data set relating to the bones based on the image; a memory unit in which at least one pattern data set is stored, the data-processing unit being programmed to adapt a respective pattern data set mathematically to the output data set and provide a static model data set relating to the bones; and a sensor unit having sensor elements that are arrangeable in a spatial relationship. The data-processing unit can provide a dynamic model data set relating to the bones based on the static model data set and information from the sensor elements. The dynamic model data set includes information about the relative position and/or mobility of the bones.

### CROSS-REFERENCE TO RELATED APPLICATIONS

This application is the United States national stage entry of International Application No. PCT/EP2022/076584, filed on Sep. 23, 2022, and claims priority to German Application No. 10 2021 124 873.6, filed on Sep. 27, 2021. The contents of International Application No. PCT/EP2022/076584 and German Application No. 10 2021 124 873.6 are incorporated by reference herein in their entireties.

### FIELD

The present invention relates to a medical technology system and a method for identifying a kinematics of bones of a patient connected to each other at a joint, in particular with respect to improving the care of the patient.

The present invention proves to be advantageous, for example, for conveying the kinematics of leg bones, in particular the femur and the tibia relative to each other, which are connected to each other via the knee joint. The present invention may also be used, for example, for identifying the kinematics of the femur relative to the pelvic bone. Further exemplary applications can be found in the area of the elbow joint, the shoulder joint and/or the spine.

### BACKGROUND

For identifying leg kinematics, for example of the knee joint and/or of the hip joint, complex systems are used today in gait laboratories, for example with infrared cameras, reflective marker elements, force plates on the floor, and fluoroscopes to identify patient-individualized, clinically usable kinematic data. Despite reliable results, the system is not suitable for use in daily clinical practice due to the considerable amount of equipment required.

### SUMMARY

The object of the present invention is to provide a medical technology system and a medical technology method of the type mentioned at the beginning, with which the kinematics of the bones can be determined in a constructively simple and preferably patient-individualized manner.

This object is solved by a medical technology system according to the invention for identifying kinematics of bones of a patient connected to each other at a joint,
comprising an imaging unit for creating at least one image of a bone arrangement comprising at least the bones in a defined orientation of the bones relative to each other, preferably in a defined viewing direction onto the bones,
a data processing unit configured and programmed to provide at least one initial dataset of the bones based on the at least one image,
a memory unit in which at least one sample data set assignable to the respective bone is stored, wherein the data processing unit is configured and programmed to computationally adapt a respective sample data set to the at least one initial dataset and to provide a patient-individualized, static model data set of the bones,
a sensor unit with respective sensor elements that may be arranged in a defined spatial relationship to the respective bone on a part of the patient's body comprising the bones,
wherein the data processing unit is configured and programmed to provide a dynamic model data set of the bones on the basis of the static model data set and based on information of the sensor elements as a result of a preferably defined movement of the bones relative to each other, wherein the dynamic model data set comprises information about the relative position and/or the relative mobility of the bones to each other.

In particular, a medical technology (kinematics detection) system for identifying kinematics of bones of a patient (or at least a first and second bone of a joint of the patient) connected to each other at a joint is proposed and provided, comprising:
an imaging unit for creating at least one (patient-specific, real) image of a bone arrangement comprising at least the bones (of the joint), in particular in a defined orientation of the bones relative to each other, preferably in a defined viewing direction onto the bones, preferably an X-ray device for creating an X-ray image of the bones of the joint,
   a data processing unit (control unit) which is adapted/configured to provide at least one digital initial dataset (with parameters) of the bones imaged in real based on the at least one image, in particular with geometric parameters for the at least first and second bones of the joint and/or density information of at least one bone of the joint, particularly preferably with geometric parameters of a length of the respective first and second bones and/or a varus and valgus angle between a femur and a tibia,
   a memory unit in which at least one (parametric bone model) sample data set is stored for the at least one first bone and the second bone of the joint, which in particular is assignable to the respective bone,
wherein the data processing unit is configured to adapt a parametric bone model from the stored sample data set with respect to at least one parameter to the at least (one parameter) of the initial dataset, in particular to the dimension of the (real) bone of the image, and thus to provide a patient-individualized three-dimensional model data set of the first and second bone,
   a sensor unit with respective sensor elements that may be arrangeable in a defined spatial relationship to the respective bone, in particular on a body part of the patient comprising the bones,
wherein the data processing unit is adapted to provide, on the basis of the patient-individualized three-dimensional model data set and based on information from the sensor elements, preferably as a result of a defined movement of the bones relative to each other, a patient-individualized, dynamic kinematics model data set of the bones, which comprises at least one piece of information about the relative position and/or the relative mobility of the bones (of the joint) with respect to each other. This dynamic (kinematics) model data set, in particular a dynamic bone model of the joint, may preferably be output via a visual display device such as an operating theater monitor, preferably with annotations, in order to simplify an evaluation of the kinematics and thus, for example, of a medical intervention. This information may also be used to simplify calibration of the sensor elements and to increase the precision of these sensor elements. By integrating patient-specific data, a dynamic model of the joint can be detected and simulated even better, for example to carry out an evaluation, e.g. for joint loads, in order to draw conclusions about implant loads and to avoid overloading the implant and to ensure a long service life and patient satisfaction.

The present invention incorporates the consideration that, in particular for identifying the kinematics of the bones connected via the joint, a movement of the bones relative to each other is required. The sensor unit, in each of which at least one sensor element is fixable to the body part comprising a bone, may preferably be used to carry out defined movements, for example under the guidance of a doctor. Information from the sensor elements may be processed by the data processing unit to form a patient-individualized dynamic model data set, in particular to determine the relative mobility of the bones and/or their relative position to each other. For identifying the static patient-individualized model data set, initially at least one image of the bones and/or a bone arrangement comprising the bones may be created in a constructively simple manner. By computationally adapting at least one predetermined sample data set assignable to a respective bone, a patient-individualized static model data set can be provided in a constructively simple manner in this way.

The patient-individualized dynamic model data set may, for example, be used as the basis for a later treatment of the patient and may be used to simplify the selection of a suitable implant.

The relative position in the dynamic model data set may, for example, comprise at least one of the following:
an angle at which the bones are oriented to each other in a static posture, with axes defined by the bone, for example, being oriented at the angle;
the pose of a joint center of the joint between the bones;
the pose of a joint center of a joint via which one of the bones is connected to another bone.

The relative mobility of the dynamic model data set may preferably comprises at least one of the following:
at least one angle circumference under which the bones are movable relative to each other at the joint;
at least one angle circumference under which a bone is movable via a joint on another bone.

In a preferred embodiment, the imaging unit is, for example, an X-ray unit, and the at least one initial dataset preferably comprises a two-dimensional representation of the bones. In this way, the initial dataset can be created in a constructively simple manner. When examining a leg, for example, an X-ray image may be created in frontal view and/or in side view (laterally) and subsequently analyzed by the data processing unit.

It is conceivable that a CT unit is used as the imaging unit, wherein the at least one initial dataset comprises a three-dimensional representation of the bones. Compared to the CT unit, however, the X-ray unit with a two-dimensional representation of the bones proves to be advantageous due to the reduced radiation exposure.

Advantageously, the data processing unit is configured and programmed to computationally determine characteristic landmarks and/or a joint center of the joint in the at least one initial dataset. Preferably, the corresponding information may be stored in the at least one initial dataset. Valuable, usable information for the subsequent dynamic model data set may already be obtained on the basis of the at least one initial dataset.

For the same purpose, it is beneficial if the system has a display unit for displaying a graphical representation of the at least one initial dataset, in particular the representation of the bone, and an input unit for a user, and if characteristic landmarks and/or a joint center of the joint are settable and/or changeable, for example correctable, by the user at the input unit. Favorably, corresponding information is storable in the at least one initial dataset.

The above-mentioned advantageous embodiments may, for example, provide for an application of the knee joint that its joint center and/or anatomical landmarks such as epicondyles on the femur, the trochanter on the femur and/or the hip center on the femur are already determined.

Based on the above-mentioned estimates and/or measurements, estimates and/or measurements can advantageously already be made on the at least one initial dataset. Preferably, in an application for the knee joint, a varus and valgus angle between the femur and tibia may be determined and/or the length of these two bones. In the case of X-ray images, however, these estimates and/or measurements may be limited to only two dimensions.

The at least one initial dataset may, in particular for the above-mentioned estimates and/or measurements, comprise a scale, wherein the data processing unit is configured and programmed to determine axes defined by the bones and/or an angle between the bones and preferably to store them in the at least one initial dataset. For example, the angle is defined by the axes.

It is advantageous if a plurality of sample data sets assignable to a respective bone is stored in the memory unit, and if the data processing unit is configured and programmed to identify the most suitable sample data set from the plurality of sample data sets using a static form model and to adapt it to the bone. In relation to a respective bone, preferably several and in particular a plurality of sample data sets may be stored. It is beneficial if the most suitable sample data set can be selected and adapted computationally. A corresponding algorithm, which is executed by the data processing unit, may, for example, take into account at least one of the characteristics of the patient listed below, which are not exhaustive: age, height, gender, medical history, ethnicity, socio-cultural background.

It is advantageous if the sample data set comprises a three-dimensional representation of the bone and if the data processing unit is configured and programmed to provide a three-dimensional static model data set and, based on this, a three-dimensional dynamic model data set. In this way, a three-dimensional patient-individualized model data set can be generated, which in particular comprises spatial information about the bones and the position of the bones relative to each other.

In the case of the exemplary application of the knee joint with femur and tibia, for example, mechanical and/or anatomical axes, an epicondylar axis, the knee center and, taking into account the pelvic bone, for example, the hip center may be determined.

Preferably, the spatial position of the femur, tibia and possibly the pelvis in relation to each other in an upright, standing position can be determined for the application for the knee joint. It is possible, for example, to determine the varus and valgus angle in a standing position and thus the orientation of bony structures in the frontal plane, but also their orientation and position in relation to each other in a sagittal plane.

It is advantageous if the sample data set comprises information about characteristic landmarks of the bone and the data processing unit is configured and programmed to store, in the static model data set, information about axes defined by the bones, dimensions of the bones, in particular their lengths, characteristic landmarks, joint centers between the bones and/or an angle between the bones.

It is beneficial if the sensor elements comprise a fastening device or if the system comprises such a fastening device assigned to them, wherein the sensor elements may be attached non-invasively to the body parts comprising the bones via the fastening device. Each sensor element may have assigned its own fastening device. The non-invasive fastening option minimizes trauma to the patient during the procedure. It is beneficial, for example, to achieve compression of soft tissue above the bone in such a way that the sensor element is as stationary as possible in relation to the bone in order to define a valid reference.

When used on the femur, for example, the sensor element is attached in the area of the epicondyles on the thigh. In the case of the tibia, the sensor element may be attached close to the knee or far down near the ankle joint, preferably on the anterior edge of the tibia.

Preferably, the sensor elements comprise an acceleration sensor, wherein the data processing unit is configured and programmed to determine a range of motion of a bone based on a signal of the acceleration sensor, taking into account the time. The acceleration values of the acceleration sensor may be used to determine the distance traveled by the acceleration sensor in terms of amount and preferably in terms of direction. Using this information, the data processing unit can identify a movement of the bones and, in particular, of the bones relative to each other.

In particular, an IMU sensor (IMU, Inertial Measurement Unit) is used as a sensor element.

In particular, a first IMU sensor may be rigidly arranged relative to a first bone of the joint and a second IMU sensor may be rigidly arranged relative to a second bone of the same joint, for example a first IMU sensor on the tibia and a second IMU sensor on the femur.

In particular, the data processing unit may be adapted to adjust the static bone model to the landmarks and dimensions set in the image. This results in a three-dimensional, patient-specific bone model in which, for example, axes such as mechanical and/or anatomical axes, in particular epicondylar axes and/or a knee center and/or a hip center and/or a pelvic inclination can be determined and can be used for an assessment, for example.

The sensor elements may be coupled to the data processing unit, for example wirelessly, in order to achieve a constructively simple embodiment.

It is beneficial if the system comprises a notice unit, in particular an optical display unit, on which notices for the execution of characteristic movements by the patient can be output. For example, the patient may perform the characteristic movements specified on the notice unit under the guidance of a user such as a doctor or independently.

When performing the movements, the data processing unit preferably incorporates the information provided by the sensor unit in the determination of the relative position and/or of the relative mobility of the bones. In particular, the movements are indicative of a condition of the bone and of the joint, especially of the kinematics of the bones via the joint. For example, a stored program may be executed in the data processing unit which, in particular via a workflow, successively provides notices for executing various types of movements. For each movement, information provided by the sensor elements can be evaluated by the data processing unit.

The movements may be mandatory or optional.

The user and/or the patient may, for example, be guided through the notices via a workflow. It may be provided that the data processing unit is transferred to a condition upon actuation of an actuating element, in which it is ready to receive the data from the sensor elements.

It is advantageous if the data processing unit is configured and programmed to identify and store its spatial relationship of the sensor elements to the bones in the dynamic model data set on the basis of information of the sensor elements. The position of the sensor elements on the body parts comprising the physical bones may be virtually transferred to the dynamic model data set. In this way, movements of the sensor elements can be mapped directly in the dynamic model data set.

Preferably, it is possible to use the information of the sensor elements to implement a three-dimensional movement image of the bones in the dynamic model data set. In a sense, the bones in the dynamic model data set can be moved in the same way as the physical bones, wherein the model data set is also supplemented with information such as axial pose, length of bones, joint centers, range of motion, angle, etc.

It is advantageous if the data processing unit is configured and programmed to determine the axes of the bones and an intersection of the axes on the basis of information of the sensor elements and to relate them to the static model data set in such a way that the axes of the bones contained therein are superimposed with the axes determined on the basis of the information of the sensor elements. In this way, the sensor elements can be in a sense calibrated in the model data set. The axes determined via the sensor elements intersect, allowing a joint center to be determined. The respective axes can be superimposed on the axes contained in the static model data set. In a three-dimensional static model data set, preferably with a scale, the length of the bones (defined via the axes), for example, can be used to calibrate the sensor elements.

Based on this, for example, a pose of the axes of the bones, an intersection of the axes and a length of the bones are stored in the dynamic model data set.

In the case of a leg, for example, the knee joint, the length of the femur and of the tibia can be determined. Using information from the pelvic bone, for example, the position of the hip joint center and, in the case of ankle bones, the ankle joint center can be determined.

Preferably, a virtual model of the bones is obtained, to which the virtual equivalents of the sensor elements are attached, so to speak. In a way, a digital image (‘twin’) of the sensor elements is created on the bones, for example the leg bones.

As already mentioned, the bones may be the femur and tibia of the patient. Preferably, at least one of the following pieces of information is stored in the dynamic model data set:
mechanical and/or anatomical femur axis and/or tibia axis;
position of the knee-joint center;
position of the hip-joint center;
position of the ankle-joint center;
varus-valgus pose of the femur and tibia;
information on the range of motion for flexion and/or extension;
information on internal and/or external rotation of femur and tibia;
information on the translation of the tibia to the femur;
information on the step length. By additionally taking time into account, for example, a movement image of the patient can be created, whereby the speed and the distance traveled are determined.
information on characteristic landmarks, for example of epicondyles and/or trochanters.

In a different application, for example, one bone is the femur and the other bone is the pelvic bone. The sensor elements are positioned, for example, on the greater trochanter of the femur and on the sacroiliac joint or the anterior superior spinae of the pelvic bone. In this way, for example, the pelvic tilt can be tracked and recorded in different situations and during different movements.

It may be provided that the data processing unit is configured and programmed to compare the dynamic model data set with an experimentally obtained measurement data set, for example on the basis of a gait analysis in a gait laboratory, to identify any deviations and to provide the user with relevant information at a notice unit.

The information obtained via the inventive system may thus be checked for plausibility with a model data set. The notices can be used to inform a user, for example, about the degree of matching and/or any deviations. For example, it is possible to train the system with regard to an improved match.

As mentioned at the beginning, the present invention also relates to a method.

A medical technology method according to the invention for identifying the kinematics of bones of a patient connected to each other at a joint, and which solves the object mentioned at the beginning:
creating at least one image of a bone arrangement comprising at least the bones in a defined orientation of the bones relative to each other, preferably in a defined viewing direction on the bones;
providing, based on the at least one image, at least one initial dataset of the bones, via a data processing unit;
   adapting, computationally via the data processing unit, of a sample data set stored in a memory unit and assignable to the respective bone, and providing a patient-individualized static model data set;
   providing a dynamic model data set of the bones, which comprises information about the relative position and/or the relative mobility of the bones to each other, on the basis of the static model data set and based on information from sensor elements as a result of a preferably defined movement of the bones relative to each other.

The advantages already mentioned in connection with the explanation of the system according to the invention can also be achieved when implementing the method. In this respect, reference is made to the above-mentioned explanations.

Advantageous configuration examples of the method according to the invention result from advantageous embodiments of the device according to the invention.

The objects of the present invention are solved with respect to a computer-readable storage medium in that this storage medium comprises commands which, when executed by a computer, cause the computer to perform the steps of the method according to the present invention.

The objects are solved with respect to a computer program in that the program comprises commands which, when executed by a computer, cause the computer to perform the steps of the method according to the present invention.

### BRIEF DESCRIPTION OF THE DRAWING FIGURES

The following description of a preferred embodiment of the invention, in conjunction with the drawing, serves to explain the invention in more detail. With the preferred embodiment of the system according to the invention, a preferred configuration example of the method according to the invention can be carried out.

FIG. 1 shows a schematic representation of a medical technology system according to the invention in a preferred embodiment for carrying out a preferred configuration example of the method according to the invention;

FIG. 2 shows a schematic representation of an image of bones, in this case femur and tibia in a bone arrangement, further comprising the pelvic bone and the foot bones;

FIG. 3 shows schematic representations of the femur and tibia in the initial dataset from the front (left) and from the side (lateral, right);

FIG. 4 shows schematic representations of bones in sample data sets stored in a memory unit of the system;

FIG. 5 shows a representation of a bone arrangement in a static model data set from the front as well as exemplary markings for determining a varus and valgus angle;

FIG. 6 shows a representation according to FIG. 5 from the side to determine a flexion angle;

FIG. 7 shows the bones in the static model data set and sensor elements of a sensor unit of the system in schematic representation; and

FIGS. 8 to 15 show pictograms as notices for the execution of movements by the patient, wherein the pictograms can be displayed on a display unit of the system.

### DETAILED DESCRIPTION

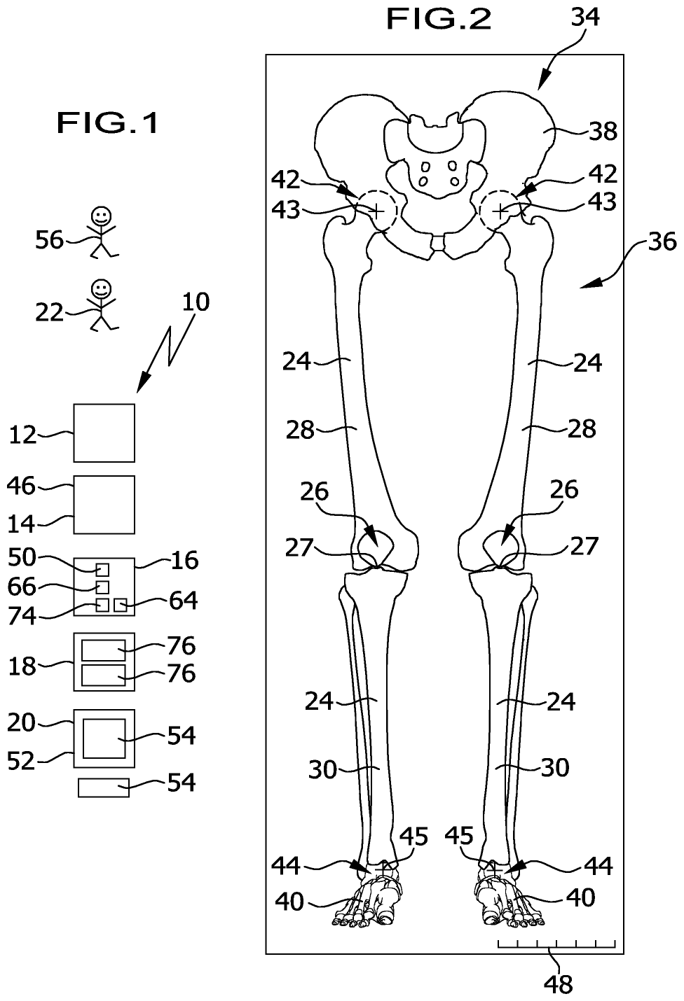

FIG. 1 shows a schematic representation of a generally advantageous embodiment of a medical technology system according to the invention, marked with the reference sign 10. The system 10 can be used to carry out a method according to the invention in a preferred embodiment.

The system 10 comprises a data processing unit 12, an imaging unit 14, a memory unit 16, a sensor unit 18 and a notice unit 20. The data processing unit 12 is coupled to the units 14, 16, 18 and 20 for transmitting information and/or signals. It is conceivable that two or more of the units 12, 14, 16, 18 and 20 are spatially and/or functionally integrated into each other.

The system 10 makes it possible to examine the kinematics of a patient's bone 22. This serves in particular to identify patient-individualized information for a later treatment, for example for implantation of an implant, in particular for implant selection and/or implantation technique.

As already described above and as will be explained below, the bones are detected via the imaging unit 14, a corresponding initial dataset is created, a static model data set is created based on a sample data set and a dynamic model data set is created based on this with the aid of the sensor unit 18. The model data sets are also patient-individualized like the initial dataset.

In the present case, the femur 28 and the tibia 30 serve as bones 24, which are articulated to each other at a joint 26, the joint 26 being the knee joint. However, as already explained above, the invention is not limited to this. Via the system 10, in particular the relative position of the femur 28 and tibia 30 and their relative mobility can be determined.

The imaging unit 14 in the present case is an X-ray unit 46. Images of a bone arrangement 34 can be created via the X-ray unit 46. In the present case, the images 36 each comprise a scale 48.

FIG. 2 shows an image 36 in which the bone arrangement 34 shows not only the femur 28 and the tibia 30 but also the pelvic bone 38 and the foot bones 40. Both legs are shown in each case, i.e. the image 36 comprises two femurs 28, two tibias 30 and two foot bones 40. In addition, the hip joint 42 and the ankle joint 44 are shown in the image 36. The joints 26, 42 and 44 each comprise a joint center 27, 43 and 45 respectively.

FIG. 2 shows an example of a frontal image 36 of the bone arrangement 34. Furthermore, at least one image 36 is preferably created from the side (lateral).

The data processing unit 12 is configured and programmed to create a patient-individualized initial dataset 50 based on the images 36. The initial dataset 50 comprises in particular at least one two-dimensional representation of the bones 24, in the present example also of the other bone arrangements 34.

In the present case, the notice unit 20 is designed in particular as a display unit 52, which comprises a controllable image display 54. Graphic representations of the bones 24 can be displayed to a user 56, in particular a doctor, on the display unit 52.

Characteristic landmarks and/or a joint center, in particular the joint center 27, may be specified by the user 56 based on the representation at an input unit 58 and information relating thereto may be stored in the initial dataset 50.

Furthermore, it may preferably be provided that the data processing unit 12 itself determines information, for example about the landmarks and/or the joint center 27, without the intervention of the user 56, and stores the relevant information in the initial dataset 50.

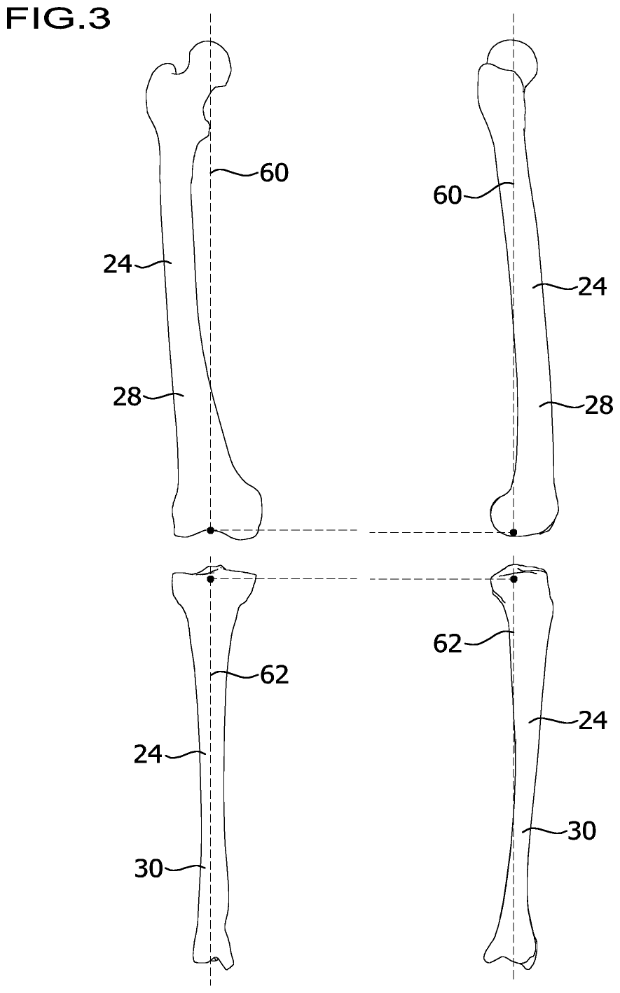

Based on the scale 48, for example, axes of the bones 24 can be determined and/or an angle between the bones 24 can be determined and stored in the initial dataset 50. This may be, for example, the mechanical femur axis 60 and the mechanical tibia axis 62, as shown schematically in FIG. 3. This enables the data processing unit 12 to already determine a varus and valgus angle based on the axes 60, 62 (not shown in FIG. 3).

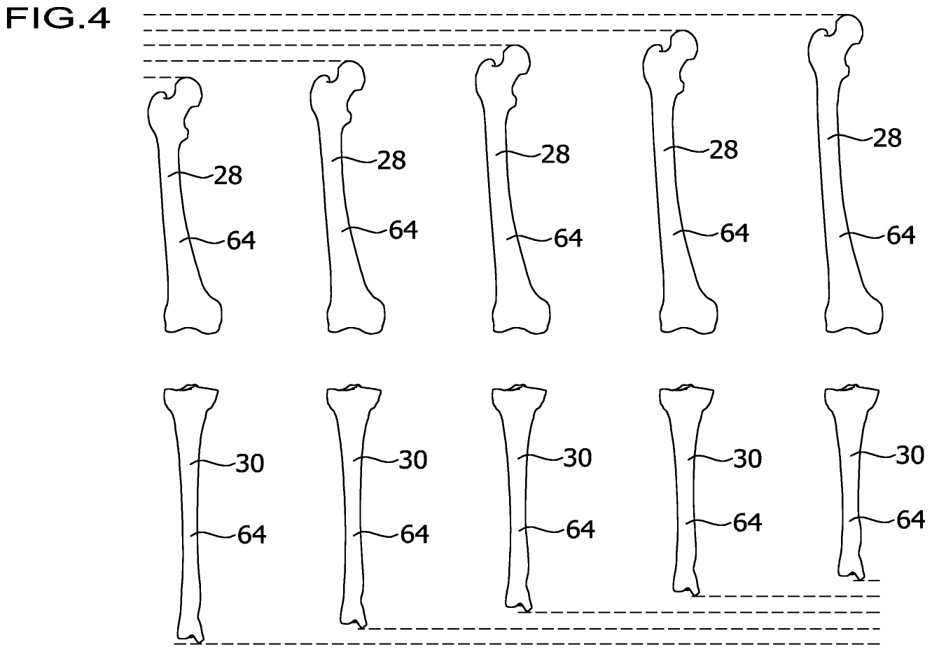

The representations of the bones 24 in the initial dataset 50 are two-dimensional. In order to create a three-dimensional patient model, the data processing unit 12 uses sample data sets 64 that are stored in the memory unit 16. FIG. 4 shows an example of a plurality of sample data sets 64 for the femur 28 and the tibia 30.

The sample data sets differ in terms of size and/or shape, for example, and in particular other criteria may be used to differentiate the sample data sets, such as gender, age, medical history, socio-cultural background, ethnicity, etc.

The sample data sets 64 each comprise a three-dimensional representation of the bones 24.

The data processing unit 12 is configured and programmed to computationally adapt the most suitable sample data set 64 to the femur 28. In a corresponding manner, the most suitable sample data set 64 is computationally adapted to the tibia 30. It is understood that this refers to femur 28 and tibia 30 in the initial dataset 50.

The data processing unit 12 creates a static model data set 66 based on the data sets 50 and 64. The model data set 66 is patient-individualized and comprises, in particular, a three-dimensional representation of the bones 24.

In the model data set 66, for example, various axes and joint centers as well as characteristic landmarks can be determined and derived.

From the adaptation of the model data sets 64 to the initial dataset 50, an estimate of the spatial position of the femur 28, tibia 30 and, if applicable, pelvic bone 38 and/or foot bones 40 in relation to each other can be estimated in particular.

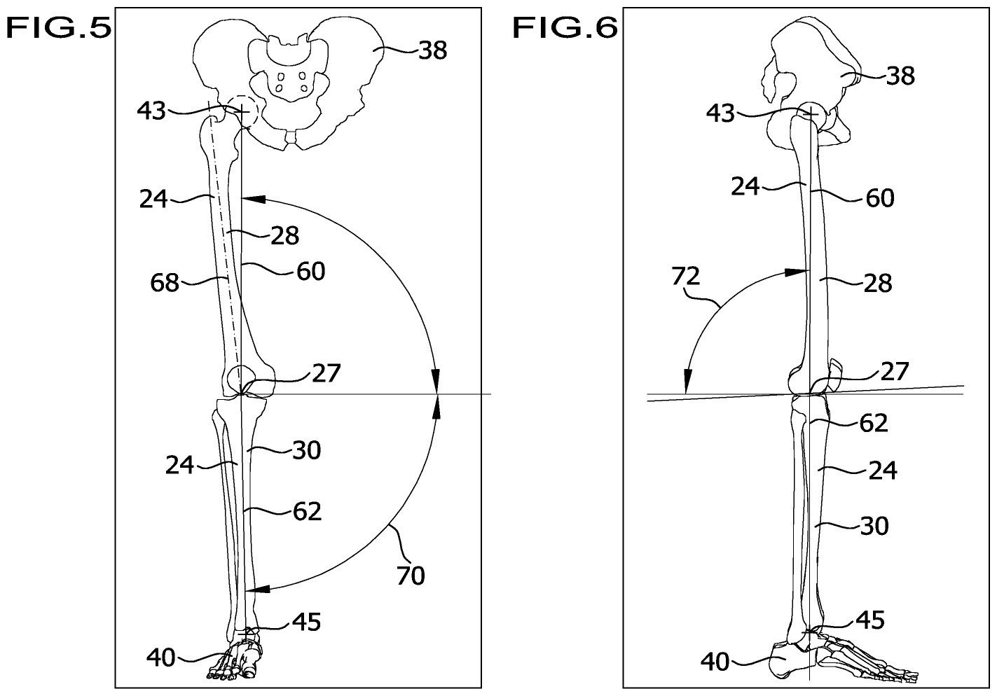

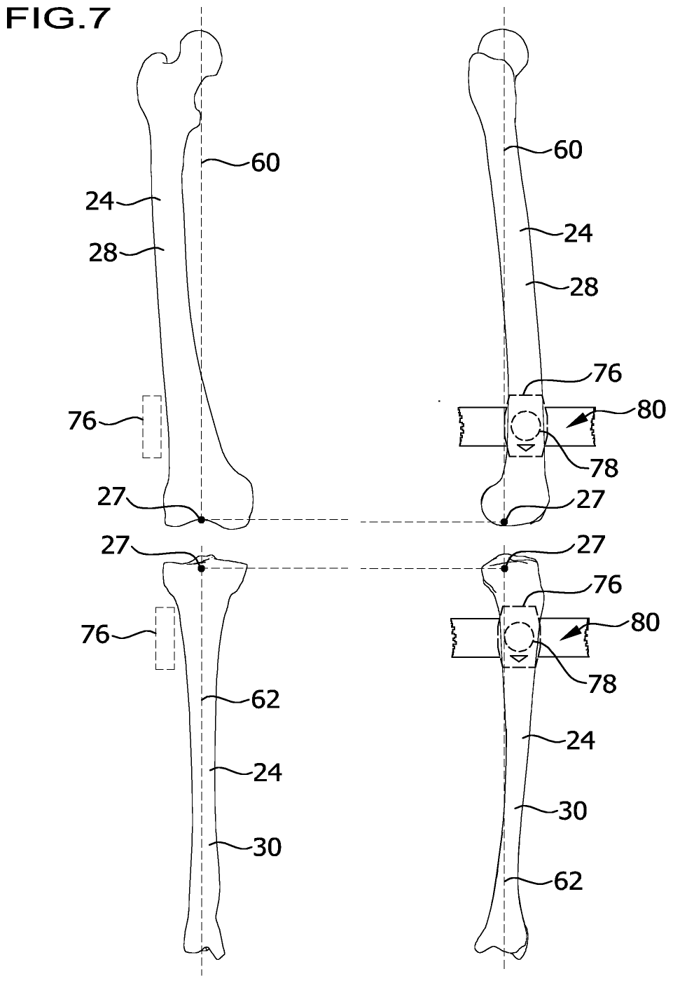

FIGS. 5 and 6 show examples of this based on a frontal view (FIG. 5) and a side view (FIG. 6). In each case, however, the illustrations are based on the three-dimensional representation of the bones 24 in the static model data set 66.

For example, FIG. 5 shows the mechanical femur axis 60 and the mechanical tibia axis 62, from which a varus and valgus angle 70 can be determined. FIG. 2 also shows the anatomical femur axis 68.

FIG. 6 shows an example of an existing flexion angle 72 between the mechanical femur axis 60 and the mechanical tibia axis 62.

The joint center 27 can also be determined in the static model data set 66. It is also possible to determine the respective length of femur 28 and tibia 30 based on scale 48.

This makes it possible, for example, to also determine the joint center 43 of the hip joint 42 and/or the joint center 45 of the ankle joint 44.

It may be possible, for example on the basis of the flexion angle 72, to determine a possible stretch deficit in the three-dimensional model in the static model data set 66. If necessary, a measurement with a protractor (e.g. goniometer) can also be used to check the calculated value.

FIGS. 5 and 6 each show only the bones 24 of one leg of the patient 22. It is understood that the model data set 66 can have three-dimensional representations for both legs as well as the corresponding information about geometries and/or characteristic landmarks. The bones 24 of the left leg in this case are not shown in FIGS. 5 and 6 for the sake of clarity.

The data processing unit 12 can computationally determine a dynamic model data set 74 from the static model data set 66 using information from the sensor unit 18. The dynamic model data set 74 comprises, in particular, a three-dimensional representation of the bones 24. In addition, at least one of the following pieces of information is stored in the dynamic model data set 74:
mechanical and/or anatomical femoral axis 60, 68 and/or tibial axis 62;
position of the knee-joint center 27;
position of the hip-joint center 43;
position of the ankle-joint center 45;
varus-valgus posture of femur 28 and tibia 30;
information on the range of motion for flexion and/or extension;
information on internal and/or external rotation of femur 28 and tibia 30;
information on the translation of the tibia 30 to the femur 28;
information on the step length;
information on characteristic landmarks, for example of epicondyles and/or trochanters.

In this way, it is possible to use the system 10 to obtain information about the relative position and relative mobility of the bones 24—in particular the femur 28 and tibia 30. A three-dimensional kinetic model is available, which also preferably comprises information about the pelvic bone 38 and/or the foot bones 40 as well as the joint centers 43 or 45.

Based on this, for example, the patient 22 can be treated individually on the basis of the patient-individualized dynamic model data set 74.

The sensor unit 18 comprises two sensor elements 76, for example IMUs (Inertial Measurement Unit), each with at least one acceleration sensor 78.

A respective sensor element 76 comprises a fastening device 80. Via the respective fastening device 80, the sensor element 76 can advantageously be fixed non-invasively to a body part comprising the bone 24. For example, a sensor element 76 is fixed non-invasively to the thigh (not shown in the drawing) and a sensor element 76 is fixed non-invasively to the lower leg (not shown in the drawing).

Preferably, the sensor elements 76 are fixed in such a way that a valid reference to the respective bone 24 can be achieved by preventing soft tissue movement as far as possible. For this purpose, the fastening device 80 can be designed as a strap or bandage, for example, which causes compression of the soft tissue and thereby secures the sensor element 76 essentially free of movement relative to the bone 24.

Information from the sensor elements 76 is detected by the data processing unit 12 and, based on this, the dynamic model data set 74 is calculated from the static model data set 66.

In particular, it is possible to calibrate the sensor elements 76 to a certain extent on the basis of the model data set 66. For example, as a result of the movement of the sensor elements 76, axes (for example the axes 60 and 62) as well as their intersection and an angle between these axes can be determined, whereby the spatial relationship to the corresponding axes and angles in the static model data set 66 for generating the dynamic model data set 74 is carried out by the data processing unit 12. For example, the axes 60, 62 determined via the sensor elements 76 can be made to coincide with the corresponding axes 60, 62 in the model data set 66. Based on the respective length of the bones 42, the sensor elements 76 can be calibrated in this way in the measuring system of the sensor unit 18.

Conversely, a spatial relationship between the sensor elements 76 and the bones 24 can be determined and stored in the dynamic model data set 74. In a sense, virtual equivalents of the sensor elements 76 are stored in the model data set 74.

With a view to recording data as completely and reliably as possible, it can be advantageous if the data processing unit 12 is used to instruct the user 56 and/or the patient 22 via corresponding notices, for example at the display unit 52. For this purpose, the data processing unit 12 can, for example, execute a workflow via a program that successively outputs a plurality of notices.

The movements themselves can be mandatory or optional. For example, a selection can be made by the user 56 and/or the patient 22.

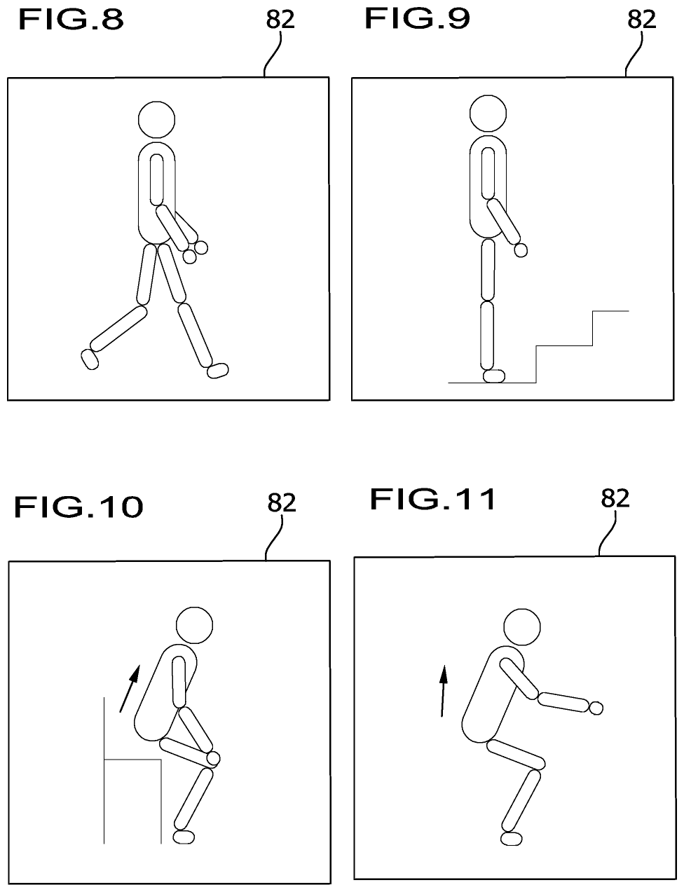

FIGS. 8 to 15 show exemplary pictograms 82, which are used to illustrate which movement is to be carried out. In particular, the pictograms 82 can be displayed on the display unit 52.

The pictogram 82 shown in FIG. 8 suggests a movement in which the patient 22 walks straight ahead for a few meters, for example.

For example, the pictogram 82 shown in FIG. 9 suggests climbing a predetermined number of steps and then descending again.

For example, the pictogram 82 shown in FIG. 10 suggests that the patient 22 should sit down and stand up again.

For example, the pictogram 82 shown in FIG. 11 suggests that the patient 22 perform a predetermined number of squats, in particular to image flexion and/or extension.

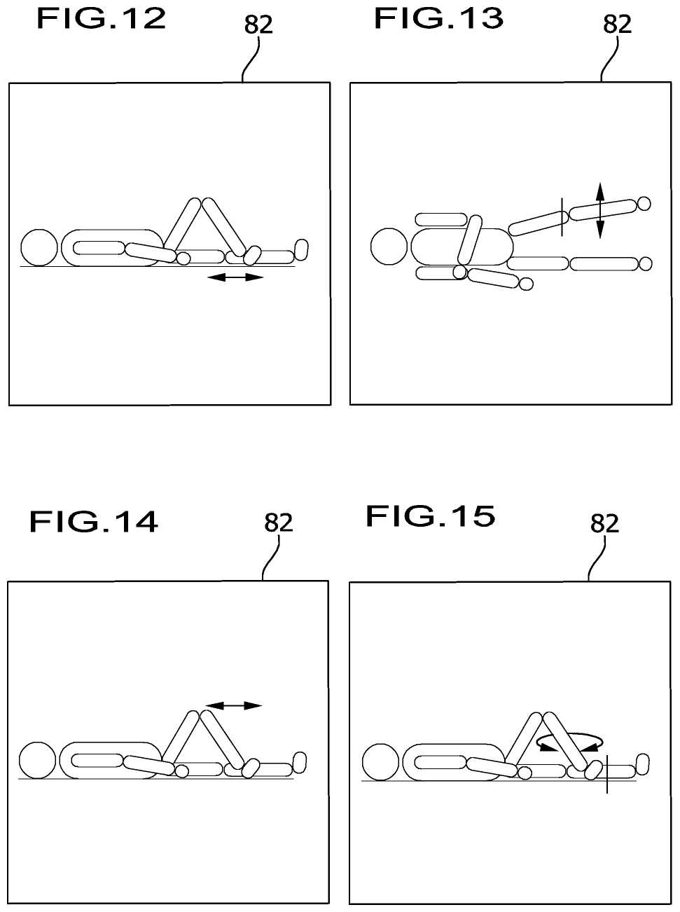

For example, the pictogram 82 in FIG. 12 suggests moving the knee from maximum extension to flexion.

For example, the pictogram 82 shown in FIG. 13 suggests loading the knee laterally and medially to identify the patient's maximum varus and valgus.

For example, the pictogram 82 shown in FIG. 14 suggests pulling the tibia maximally forward or pushing it backward.

For example, the pictogram 82 shown in FIG. 15 suggests rotating the tibia internally and externally to the maximum possible angle.

1.-21. (canceled)

22. A medical device system for identifying kinematics of bones of a patient connected to each other at a joint, the medical device system comprising:
an imaging unit for creating at least one image of a bone arrangement comprising at least the bones in a defined orientation of the bones relative to each other;
a data processing unit configured and programmed to provide at least one initial dataset of the bones based on the at least one image;
a memory unit in which at least one sample data set assignable to a respective bone is stored; and
a sensor unit with sensor elements that are arrangeable in a defined spatial relationship to the respective bone on a part of the patient comprising the bones,
the data processing unit being configured and programmed to computationally adapt a respective sample data set to at least one initial dataset and to provide a static model data set of the bones that is patient-individualized,
the data processing unit being configured and programmed to provide a dynamic model data set of the bones based on the static model data set and based on information of the sensor elements as a result of a movement of the bones relative to each other, wherein the dynamic model data set comprises information about a relative position and/or a relative mobility of the bones to each other, and
the relative position comprising at least one of:
an angle at which the bones are oriented to each other in a static posture;
a pose of a joint center of the joint between the bones; and
a pose of a joint center of a joint via which one of the bones is connected to another bone.

23. The medical device system according to claim 22, wherein the relative mobility comprises at least one of:
at least one angle circumference under which the bones are movable relative to each other at the joint; and
at least one angle circumference under which a bone is movable via a joint on another bone.

24. The medical device system according to claim 22, wherein the imaging unit is an X-ray unit and the at least one initial dataset comprises a two-dimensional representation of the bones.

25. The medical device system according to claim 22, wherein the data processing unit is configured and programmed to computationally determine characteristic landmarks and/or a joint center of the joint in the at least one initial dataset and to store corresponding information in the at least one initial dataset.

26. The medical device system according to claim 22, further comprising a display unit for displaying a graphical representation of the at least one initial dataset and an input unit for a user, and in that characteristic landmarks and/or a joint center of the joint are settable and/or changeable by the user at the input unit, and in that corresponding information is storable in the at least one initial dataset.

27. The medical device system according to claim 22, wherein the at least one initial dataset comprises a scale and in that the data processing unit is configured and programmed to determine axes defined by the bones and/or an angle between the bones and to store them in the at least one initial dataset.

28. The medical device system according to claim 22, wherein a plurality of sample data sets assignable to a respective bone is stored in the memory unit, and in that the data processing unit is configured and programmed to identify a most suitable sample data set from the plurality of sample data sets using a static form model and to adapt it to the bone.

29. The medical device system according to claim 22, wherein the at least one sample data set comprises a three-dimensional representation of the bone and in that the data processing unit is configured and programmed to provide a three-dimensional static model data set and, based on this, a three-dimensional dynamic model data set.

30. The medical device system according to claim 22, wherein the at least one sample data set comprises information about characteristic landmarks of the bone and the data processing unit is configured and programmed to store, in the static model data set, information about axes defined by the bones, dimensions of the bones, their lengths, characteristic landmarks, joint centers between the bones and/or an angle between the bones.

31. The medical device system according to claim 22, wherein the sensor elements comprise or are associated with a fastening device via which the sensor elements are non-invasively attachable to body parts comprising the bones.

32. The medical device system according to claim 22, wherein the sensor elements comprise an acceleration sensor and wherein the data processing unit is configured and programmed to determine a range of motion of a bone based on a signal of the acceleration sensor taking into account a time.

33. The medical device system according to claim 22, further comprising a notice unit on which notices for execution of characteristic movements by the patient can be output, the data processing unit incorporating the information provided by the sensor unit in determining the relative position and/or of the relative mobility of the bones when movements are executed.

34. The medical device system according to claim 22, wherein the data processing unit is configured and programmed to identify and store a spatial relationship of the sensor elements to the bones in the dynamic model data set based on information of the sensor elements.

35. The medical device system according to claim 22, wherein the data processing unit is configured and programmed to determine axes of the bones and an intersection of the axes based on information from the sensor elements and to relate the axes of the bones and the intersection of the axes to the static model data set in such a way that axes of the bones contained therein are superimposed with the axes determined based on the information of the sensor elements.

36. The medical device system according to claim 35, wherein, based on this, a pose of the axes of the bones, an intersection of the axes and a length of the bones are stored in the dynamic model data set.

37. The medical device system according to claim 22, wherein the bones are a femur and a tibia of the patient, and wherein the dynamic model data set stores at least one of:
mechanical and/or anatomical femur axis and/or tibia axis;
position of a knee-joint center;
position of a hip-joint center;
position of an ankle-joint center;
varus-valgus pose of a femur and a tibia;
information on a range of motion for flexion and/or extension;
information on internal and/or external rotation of the femur and the tibia;
information on a translation of the tibia to the femur;
information on a step length; and
information on characteristic landmarks.

38. The medical device system according to claim 22, wherein the data processing unit is configured and programmed to compare the dynamic model data set with an experimentally obtained measurement data set to identify any matches and/or deviations and to provide a user with relevant information at a notice unit.

39. A medical technology method for identifying kinematics of bones of a patient connected to each other at a joint, comprising:
creating at least one image of a bone arrangement comprising at least the bones in a defined orientation of the bones relative to each other;
providing, based on the at least one image, at least one initial dataset of the bones, via a data processing unit;
adapting, computationally via the data processing unit, of a sample data set stored in a memory unit and assignable to the respective bone, and providing a patient-individualized static model data set;
providing a dynamic model data set of the bones, which comprises information about a relative position and/or a relative mobility of the bones to each other, based on the static model data set and based on information from sensor elements as a result of a movement of the bones relative to each other.

40. A computer-readable storage medium comprising commands which, when executed by a computer, cause the computer to perform steps of claim 39.

41. A computer program comprising commands which, when the computer program is executed by a computer, cause the computer to perform steps of claim 39.
